(19) United States
(12) Patent Application Publication
(10) Pub. No.: US 2025/0228495 A1
(43) Pub. Date: Jul. 17, 2025

(54) REAL TIME EVALUATING A MECHANICAL MOVEMENT ASSOCIATED WITH A PRONUNCIATION OF A PHENOME BY A PATIENT

(71) Applicant: SpeakSuite, Inc., Herzliya (IL)

(72) Inventors: Ben-Zion Treger, Tzur Hadassa (IL); Raphael Nassi, Herzliya (IL); Nir Geva, Ness Ziona (IL)

(73) Assignee: SpeakSuite, Inc., Herzliya (IL)

(21) Appl. No.: 18/700,660

(22) PCT Filed: Oct. 10, 2022
(86) PCT No.: PCT/IB2022/059709

§ 371 Date: Apr. 11, 2024

Related U.S. Application Data
(60) Provisional application No. 63/262,384, filed on Oct. 11, 2021.

(51) Int. Cl. A61B 5/00; A61B 5/107; G06V 10/764; G06V 40/16; G16H 50/30

(52) U.S. Cl. CPC A61B 5/4803; A61B 5/1075; A61B 5/7267; G06V 10/764; G06V 40/176; G16H 50/30

(57) ABSTRACT

A non-transitory computer readable medium for real time evaluating a mechanical movement associated with a pronunciation of a phenome by a patient, the non-transitory computer readable medium stores instructions that once executed by a processing circuit cause the processing circuit to: (i) receive visual information regarding a patient mechanical movement that is associated with the pronunciation of the phenome by the patient; (ii) apply a machine learning based extraction process for extracting features of the patient mechanical movement; (iii) determine, by a classifier and based on the features, a quality of the patient mechanical movement; wherein the classifier was trained by a training process that included feeding to the classifier with features of examples of visual mechanical movement information, wherein the examples are associated with a quality score that is determined based on speech therapy experts feedbacks; and (iv) respond to the quality of the patient mechanical movement.

### CROSS REFERENCE

This application claims priority from U.S. provisional patent Ser. No. 63/262,384 filing date Oct. 11, 2021 which is incorporated herein by its entirety.

### BACKGROUND

About 10% of the world's population suffers from some form of a speech-Language disorder. These communication difficulties result in a limited ability to participate in social, academic, or occupational environments.

Many children fail exercise at home, prolonging the treatment and making it less effective due to lack of direct clinical supervision, boredom, and difficulty following through.

There is a growing need to provide system, methods and non-transitory computer readable medium for motiving children with speech development disorders and adults with neurological brain damage to practice anywhere anytime in a fun, user-friendly interface making the training process exciting and rewarding.

### BRIEF DESCRIPTION OF THE DRAWINGS

The embodiments of the disclosure will be understood and appreciated more fully from the following detailed description, taken in conjunction with the drawings in which:

FIG. 1 shows a schematic, pictorial illustration of an example of a system for conducting speech therapy computer-assisted training;

FIG. 2 is a schematic, flow diagram of an example of a process for assessing therapy progress:

FIG. 3 is a schematic graph of an example of a therapy assessment output;

FIG. 4 is an example of a method;

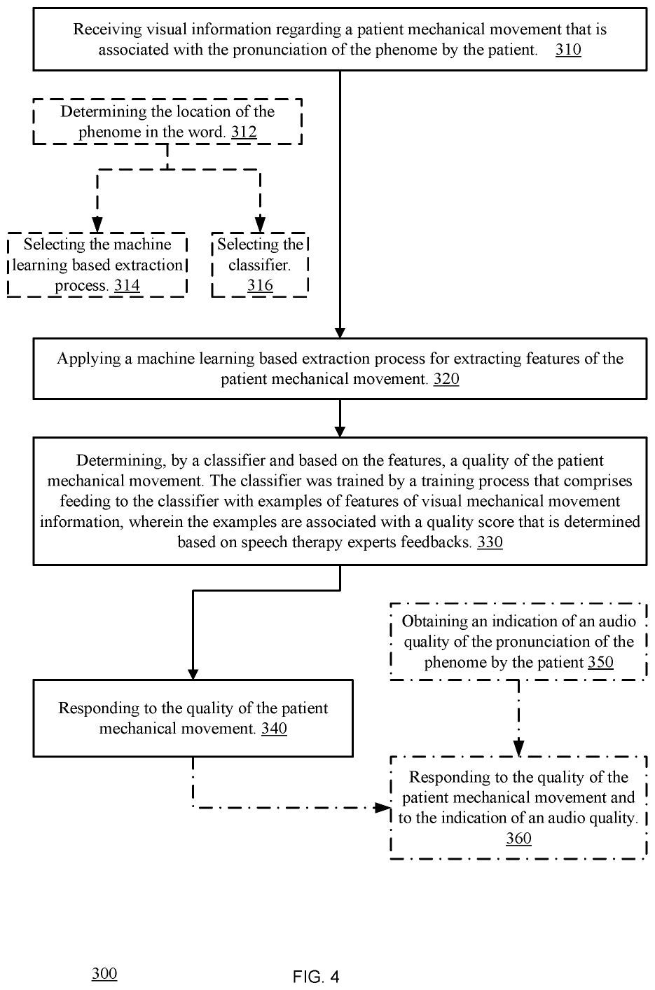

FIG. 5 is an example of a method;

FIG. 6 is an example of a computerized system and its environment,

FIG. 7 is an example of a computerized system and its environment;

FIG. 8 is an example of images with a clear portion and a distorted portion.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that can be executed by the system and should be applied mutatis mutandis to a non-transitory computer readable medium that stores instructions that once executed by a computer result in the execution of the method.

There may be provided a computerized method for real time evaluating a mechanical movement associated with a pronunciation of a phenome by a patient.

The method may be applied on any phenome. It is assumed that the phenome is selected (by the patient or by another party such as a speech therapy clinician) and that the patient is requested to pronounce words that include the phenome.

The method may be applied to different phonemes—concurrently or sequentially.

The method operates in real time to provide a response to the patient in real time—for example with a delay (from the moment the mechanical movement is executed till the provision of feedback to the patient) of less than a few seconds—for example less than 1, 2, 3, 4, 5, 6, 7, 8 seconds—and the like.

The computer implemented method is highly accurate. The accuracy may be contributed to the usage of speech therapy experts feedbacks. The number of speech therapy experts may exceed two, may be three or more. Using a uneven number of speech therapy experts may ease reaching a majority vote—but the number of speech therapy experts may be an even number. An example of a speech therapy expert is a speech therapy clinician, or any other speech therapy expert. An increase in the number of speech therapy experts may increase the accuracy. It has been found that using three speech therapy experts provided a highly accurate process.

The accuracy may also be contributed to a selection of anchors of a face of the patient that appear in visual information regarding the patient mechanical movement.

The accuracy may also be contributed to a selection of features to be extracted from the visual information. The selection aims to select features that distinguish between mechanical movements of different quality levels.

The accuracy may be contributed to the usage of a machine learning based extraction process that is followed by a classifier. Each one of the machine learning based extraction process and the classifier may be independently tuned during a training process to provide high accuracy. The separation between the machine learning based extraction process and the classifier also allows using a classifier that is not a machine learning classifier—which may be more accurate and/or consumes less resources than a machine learning classifier. The classifier may be a deep neural network classifier, a Support Vector Machine (SVM) classifier, and the like.

The method may also obtain indication about the audio quality of the phenome—in addition to the quality of the mechanical movement. Using the audio quality, in addition to the quality of the mechanical movement increases the accuracy of the method.

The method is also highly effective and significantly reduces consumption of computational and/or memory resources.

The high effectiveness may be contributed to the selection of features—out of a larger group of feature candidates.

The high effectiveness may be contributed to the separation between the machine learning based extraction process and the classifier also allows using a classifier that is not a machine learning classifier and is more effective than a machine learning classifier.

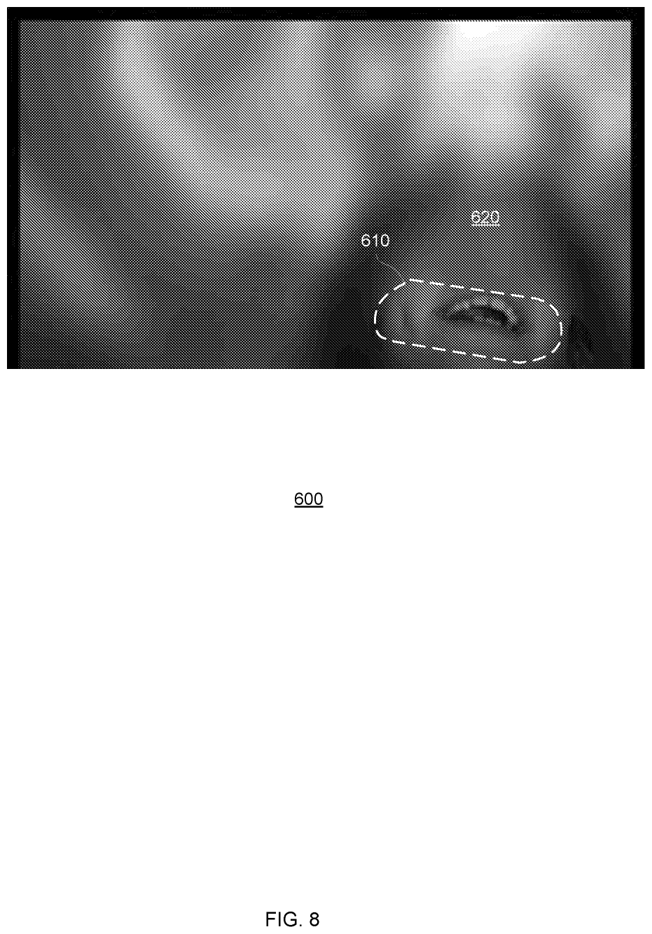

The visual information may be include clear visual information that can be processed. The clear visual information may be limited to at least a part of the mouth and at least one part of the vicinity of the mouth. The clear visual information may be small enough to prevent or to significantly reduce the chances of identifying the patient. This reduction also saves computational and/or memory resources—as other parts of the face of the patient are nor processed and/or may not be saved. FIG. 8 illustrates an example of image 600 that include a clear part 610 of the mouth and the vicinity of the mouth and an unclear portion 620.

The classifier may be trained by a training process that includes feeding to the classifier with features of examples of visual mechanical movement information, wherein the examples are associated with a quality score that is determined based on speech therapy experts feedbacks. The examples may be consensus examples in which the feedbacks of all speech therapy experts are the same. Alternatively, the examples may be majority examples in which only the majority of the feedbacks of all speech therapy experts are the same. The examples of visual mechanical movement information may also be referred to as video or video streams.

If there are enough consensus examples than using the consensus examples for training may increase the accuracy of the classifier. It there are not enough consensus examples and there are enough majority examples—then using the majority examples (in addition to or instead of the consensus examples) for training may increase the accuracy of the classifier.

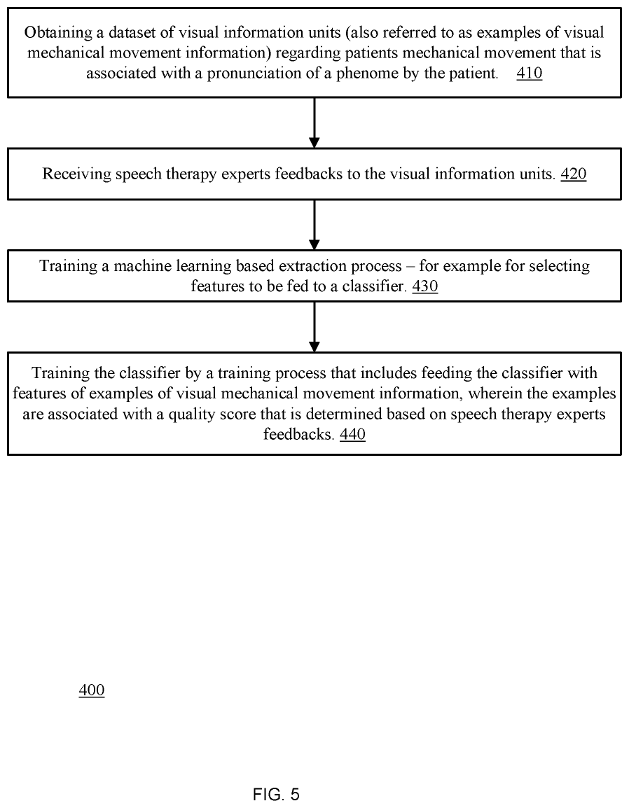

FIG. 5 illustrates an example of computer implemented method (“method”) 300 for real time evaluating a mechanical movement associated with a pronunciation of a phenome by a patient.

Method 300 may start by step 310 of receiving visual information regarding a patient mechanical movement that is associated with the pronunciation of the phenome by the patient.

The visual information may be partially masked or obscured or distorted—for example for reducing the chances of identifying the patient.

The visual information may include a clear segment of a face of the person, the clear segment covers at least a part of a mouth of the patient and at least a part of a vicinity of the mouth of the patient, the vicinity does not include eyes of the patient.

The visual information may also include an unclear representation of eyes and nose of the patient.

The visual information may be acquired by any image sensor (for example a camera of a smartphone or computer of the patient) and may be processed to provide the clear position before transmission to a computerized system that executes method 300. The computerized system may include one or more computers—such as but not limited to cloud computers, a computer of the one or more computers may be a server, a desktop computer, a laptop computer, a hardware accelerator, a supercomputer, and the like. The computerized system (for example the one or more computers) may include one or more processing circuits. A processing circuit may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits.

Step 310 may be followed by step 320 of applying a machine learning based extraction process for extracting features of the patient mechanical movement.

The features may be selected out of a larger group of feature candidates. The selection aims to select features that distinguish between mechanical movements of different quality levels.

Step 320 may be followed by step 330 of determining, by a classifier and based on the features, a quality of the patient mechanical movement. The classifier was trained by a training process that comprises feeding to the classifier with examples of features of visual mechanical movement information, wherein the examples are associated with a quality score that is determined based on speech therapy experts feedbacks.

The classifier may be a machine learning classifier. The classifier may differ from a machine learning classifier.

The examples may be consensus examples that received a same speech therapy experts feedback from all speech therapy experts.

The examples may be majority examples that received a same speech therapy experts feedback only from a majority of speech therapy experts.

Step 330 may be followed by step 340 of responding to the quality of the patient mechanical movement.

The responding may include participating in a transmission of mechanical movement feedback to the patient. Participating may mean transmitting the mechanical movement feedback, sending a request or a command to a communication unit to perform the transmitting, storing the mechanical movement feedback to the patient in a memory and initiating the transmission.

The mechanical movement feedback may be provided in any manner—audio, visual, audio/visual, tactile, and the like.

The mechanical movement feedback may include a patient provided quality score.

The mechanical movement feedback may also include information about how to correct the patient mechanical movement, when the patient provided quality score is indicative of a faulty mechanical movement.

The patient provided quality score may be the same score the as the score of quality of the patient mechanical movement—as determined in step 430.

Alternatively—the patient provided quality score may differ from the score the as the score of quality of the patient mechanical movement—as determined in step 430. The patient provided quality score may be less detailed that the score determined in step 430 (simpler—may be for example of fewer quality levels) and/or may be a textual score. For example—instead of a numerical score that may have K possible values—the patient provided quality score may include Q possible values—wherein K well exceeds Q (for example by a factors of 2, 3, 4, 5, 10 and even more).

According to an example, method 300 may provide an evaluation of the mechanical movement that is responsive to a location of the phenome within a word.

Accordingly—location of the phenome within a word spoken by the patient may be known in advance—as the method is aware to the words that the client is requested to speak.

In this example, method 300 also includes step 312 and at least one of steps 314 and 316.

Step 312 include determining the location of the phenome in the word.

Step 312 may be followed by step 314 of selecting the machine learning based extraction process, out of a plurality of machine learning based extraction process associated with different locations of the phenome, based on the location of the phenome within the word. Step 314 may be followed by step 320

Step 312 may be followed by step 316 of selecting the classifier out of a plurality of classifiers associated with different locations of the phenome, based on the location of the phenome within the word. Step 316 may be followed by step 330.

For simplicity of explanation step 316 is illustrated as preceding step 320.

According to an example, method 300 may provide an evaluation of the mechanical movement that is responsive to one or more additional parameters (such as age of patient and/or gender of patient and/or location of the patient and/or ethnicity of the patient). In this case method 300 may include a step that include determining the one or more additional parameter (applying, mutatis mutandis step 312), and may include one or more steps for selecting the classifier (applying, mutatis mutandis step 316) and/or selecting the machine learning based extraction process (applying, mutatis mutandis step 314).

It should be noted instead of (or in addition to) selecting a classifier—one or more parameters of the classifier may be amended.

It should be noted instead of (or in addition to) selecting a machine learning based extraction process—one or more parameters of the machine learning based extraction process may be amended.

According to an example, method 300 may also include step 350 of obtaining an indication of an audio quality of the pronunciation of the phenome by the patient.

Method 300 may also include step 360 of responding to the quality of the patient mechanical movement and to the indication of the audio quality.

Step 360 is illustrated as being separated from step 340—but may be a part of step 340.

Step 360 may include generating a combined score—that may be the patient provided quality score. Alternatively, the patient provided quality score may be determined based on the combined score—but may differ from the combined score.

The combined score may be an average of the quality of the patient mechanical movement (MQ) and to the indication of the audio quality (AQ).

The combined score may be a weighted sum (that differs from an average) of the quality of the patient mechanical movement and to the indication of the audio quality.

The following table illustrates an example of the overall score and mechanical movement feedbacks to the patient in various cases:

| | | Combined score (CS) and |
|---|---|---|
| AQ | MQ | feedback to the patient |
| Good | Good | CS = Good |
| | | Encouraging feedback |
| Good | Bad | CS = Medium |
| | | Nonjudgmental feedback |
| | | (no encouraging feedback). |
| | | Correctional feedback |
| | | only (reminder). |
| Bad | Good | CS = Medium |
| | | Nonjudgmental feedback |
| | | (no encouraging feedback). |
| | | Correctional feedback |
| | | only (reminder) |
| Bad | Bad | CS = Bad |
| | | Encouraging feedback. |
| | | Correctional feedback |

Examples of calculating CS are provided below:
a. Normalize AQ and MQ (for example to a range between zero and one)
   i. AQ_norm=AQ/(AQ+MQ).
   ii. MQ_norm=MQ/(AQ+MQ).
   iii. Determine relative weight factor for AQ and MQ—for example WF=0.6
   iv. Calculate the weighted scores:
      1. AQ_weighted=AQ_norm*(1+WF).
      2. MQ_weighted=MQ_norm*(1−WF).
      3. AQ weighted and MQ weighted may be further processed—for example by providing their values to the AQ and MQ columns of the mentioned above table.

Other calculations may be applied, other values of WF may be applied.

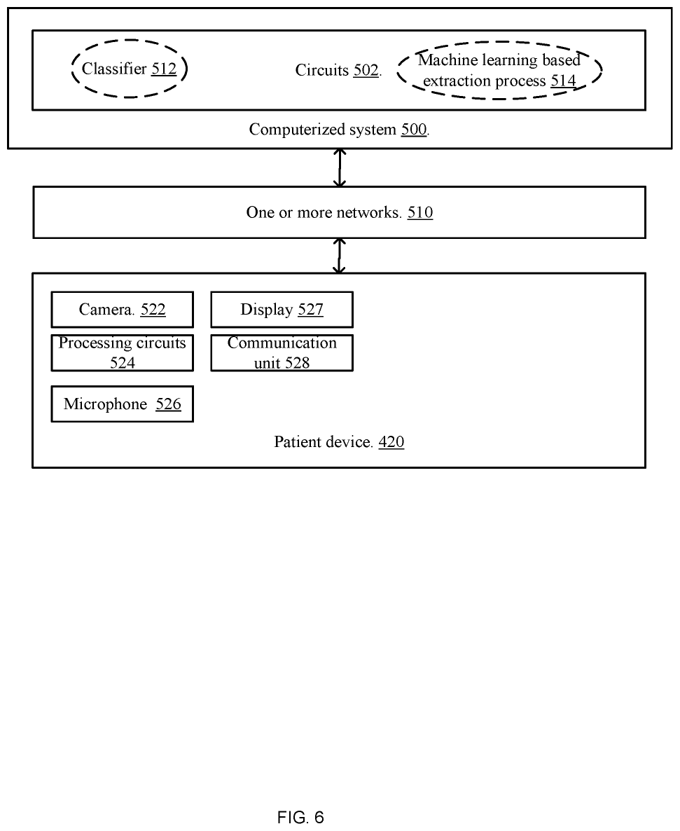

FIG. 6 illustrates an example of a training method 400.

For simplicity of explanation training method is illustrated in relation to a (certain) phenome.

The training process may be applied, mutatis mutandis, to multiple phenomes.

The training process may be applied, mutatis mutandis, in relation to one or more phenomes located at different locations within words.

The training process may be applied, mutatis mutandis, in relation to one or more phenomes having one or more additional parameters (such as age of patient and/or gender of patient and/or location of the patient and/or ethnicity of the patient).

Training method 400 may start by step 410 of obtaining a dataset of visual information units (also referred to as examples of visual mechanical movement information) regarding patients mechanical movement that is associated with a pronunciation of a phenome by the patient.

Step 410 may include rejecting visual information units that are of insufficient video quality. The insufficient quality may be determined by computerized methods (for example based on video parameters such as signal to noise ratio, based on a machine learning process trained to evaluate video quality) and/or a one or more humans.

Step 410 may include determining anchors within a clear portion of the visual information units to be tracked during method 400.

Step 410 may be followed by step 420 of receiving speech therapy experts feedbacks to the visual information units.

The visual information units are displayed to speech therapy experts and they are requested to provide feedback regarding the quality of the mechanical movements associated with the pronunciation of the phenome. The speech therapy experts may also receive the audio information about the audio generated during the pronunciation of the phenome—which may assist the speech therapy experts to provide feedback.

The outcome of step 420 may indicate that there may not be enough examples of visual information unit of a certain quality—and step 420 may be followed by applying step 410 on new visual information units. For example—there may be a need to acquire thousands of bad visual information units (visual information units that are deemed to be bad by the speech therapy experts). There may be a need to acquire thousands of good visual information units (visual information units that are deemed to be good by the speech therapy experts). The number of good visual information units may equal to the number of bad visual information units—or may differ (be smaller than or bigger than) the number of bad visual information units.

Step 420 may be followed by step 430 of training a machine learning based extraction process—for example for selecting features to be fed to a classifier. The features are selected based on their statistical significance—the ability to differentiate between visual information units of mechanical movements (associated with the pronunciation of a phenome) of different qualities—for example between good mechanical movement and bad mechanical movements.

Step 430 may include performing multiple repetitions of (a) selecting a currently evaluated set of features to be sent to a classifier, (b) feeding multiple visual information units to a machine learning based extraction process to provide multiple values of the set of features, and (c) determining features of statistical significance. The statistical significance is determined based, at least in part, on a relationship between values of the features and the speech therapy experts feedbacks.

Step 430 may include testing the currently evaluated set of features with testing visual information units—to validate their distinctiveness between mechanical movements (associated with the pronunciation of a phenome) of different qualities.

Step 430 may be followed by step 440 of training the classifier by a training process that includes feeding the classifier with features of examples of visual mechanical movement information, wherein the examples are associated with a quality score that is determined based on speech therapy experts feedbacks.

The examples may be consensus examples in which the feedbacks of all speech therapy experts are the same. Alternatively, the examples may be majority examples in which only the majority of the feedbacks of all speech therapy experts are the same. If there are enough consensus examples than using the consensus examples for training may increase the accuracy of the classifier. It there are not enough consensus examples and there are enough majority examples—then using the majority examples (in addition to or instead of the consensus examples) for training may increase the accuracy of the classifier.

Training process 400 may be applied multiple times. For example—a certain execution of training process 400 may be executed on visual information units that were obtained after an execution of a previous execution of training process 400. It is noted that instead of performing a full training process, a re-training process may be applied.

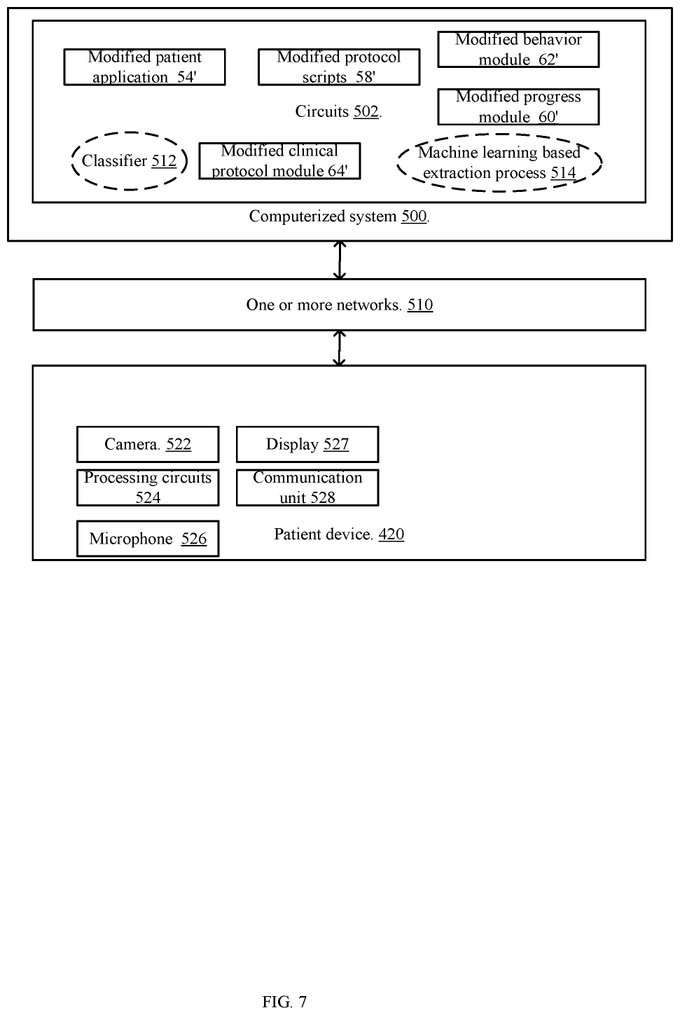

FIG. 7 illustrates an example of a computerized system 500 and its environment.

The computerized system 500 is configured to perform real time evaluating a mechanical movement associated with a pronunciation of a phenome by a patient, the computerized system comprises circuits 502 that are configured to: (i) receive visual information regarding a patient mechanical movement that is associated with the pronunciation of the phenome by the patient; (ii) apply a machine learning based extraction process for extracting features of the patient mechanical movement; (iii) determine, by a classifier and based on the features, a quality of the patient mechanical movement; wherein the classifier was trained by a training process that comprises feeding to the classifier with features of examples of visual mechanical movement information, wherein the examples are associated with a quality score that is determined based on speech therapy experts feedbacks, and (iv) respond to the quality of the patient mechanical movement.

The circuits 502 may include one or more processing circuits, memory units, communication units, and the like.

In FIG. 5 the one or more processing circuits are illustrated as hosting (or executing) classifier 512 and machine learning based extraction process 514.

The computerized system 500 may be in communication, via one or more networks 510 with one or more patient devices such as patient device 520 that may include a camera 522, one or more processing circuits 524, a microphone 526, a display 527 or other man machine interfaces (keyboard, touchscreen, speaker, tactile interface), and a patient device communication unit 528.

The camera 522 is configured to obtain visual information of the patient while pronunciation of the phenome, the one or more patient device processing circuits 524 are configured to modify the visual information to provide a clear part that include at least a part of the mouth and at least apart of the vicinity of the mouth—without including various identifying features of the patient—such as eyes and/or ears and/or noses—or parts thereof.

The patient device microphone 526 may record the audio. The patient device communication unit 528 may output the audio and/or the visual information (for example—after being modified) to the one or more networks 510. Any man machine interface of the patient device may output feedback to the patient.

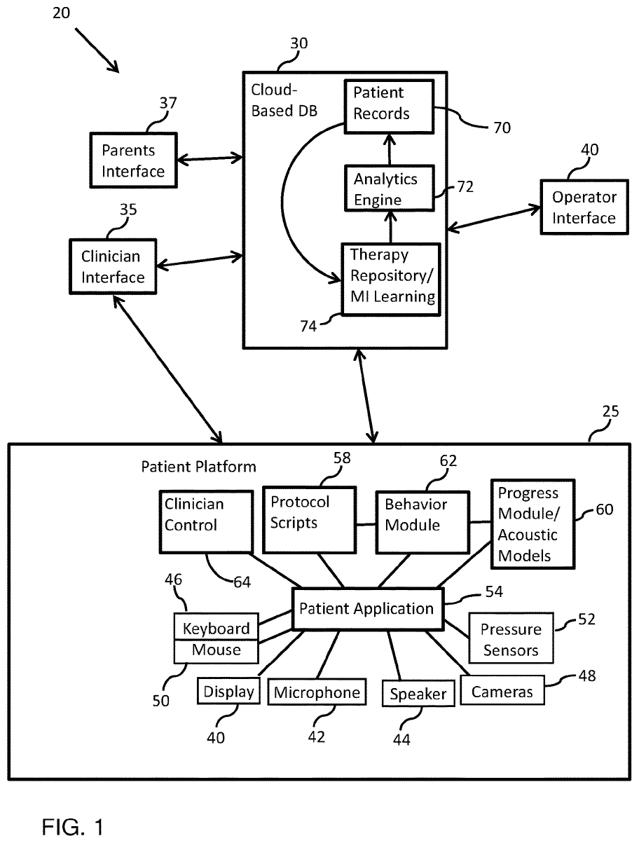

The patient device may include (or may execute) at least one of a patient application (denoted 54 in FIG. 1), clinical protocol module (denoted 64 in FIG. 1), protocol scripts (denoted 59 in FIG. 1), behavior module (denoted 62 in FIG. 1), progress module (denoted 60 in FIG. 1), and the like.

Additionally or alternatively, any one of a modified patient application, a modified protocol scripts module, a modified behavior module, and a modified progress module may belong to the computerized device and not to the client device. The modification of the model is made to apply the changes required for executing the module on the computerized device and not on the patient device and/or for adapting any of the modules to the evaluation of the mechanical movement. FIG. 7 illustrates an example of a computerized system 500 in which circuits 502 include or are configured to execute modified patient application 54′, modified clinical protocol module 64′, modified protocol scripts 59′, modified behavior module 62′, and modified progress module 60′.

Any one of the patient application, clinical protocol module, protocol scripts, behavior module, progress module may participate in the determining of audio quality—and/or may participate in the evaluating of a mechanical movement associated with a pronunciation of a phenome.

The computerized system may include the analytics engine (denoted 72 in FIG. 1). The analytic system may be modified to participate in the evaluating of a mechanical movement associated with a pronunciation of a phenome.

### An Example of a System

U.S. patent application Ser. No. 17/058,103 (publication serial number 2021/0202096) which is incorporated herein by reference in its entirety illustrates a method and system for speech therapy computer-assisted training and repository for determining scores related to audio quality.

According to an example of the current application—the method and system may be modified, mutatis mutandis, to provide real time evaluating a mechanical movement associated with a pronunciation of a phenome by a patient.

The system and method are illustrated below.

FIG. 1 shows a schematic, pictorial illustration of a system 20 for conducting speech therapy computer-assisted training, in accordance with an embodiment. System 20 may include four computer-based systems: a patient platform 25, a cloud-based, back-end database system 30, a clinician interface 35, a parent interface 37, and an operator interface 40.

The patient platform 25 system includes standard computer I/O devices, such as a touch sensitive display 40, a microphone 42, a speaker 44, a keyboard 46 and a mouse 50. In addition, the platform may have one or more cameras 48, as well as air pressure sensors 52, which may be similar to microphones, and which may provide intraoral data related to a patient's pronunciation of sounds. A speech therapy “patient” application 54 runs on the patient platform 25, driving the various output devices and receiving patient responses from the input devices during a patient therapy session. During such a session, the patient application 54 presents games or other interactive activities (collectively referred to hereinbelow as activities), which are intended to train the patient to pronounce certain sounds, words, or phrases correctly. Instructions may be given to the patient visually via the display 40 or audibly via the speaker 44, or a combination of two. The microphone 42 captures the patient's speech and conveys the captured speech signals to the patient application 54, which analyzes the sounds, words, and sentences to identify speech particles, phonemes and words.

The patient application 54 determines the activities to present to the patient, as well as the audio and visual content of each sequential step of those activities, according to a protocol, typically set by one or more protocol scripts 58, which are typically configured by the clinician. The protocol scripts are typically stored in a patient records system 70 of the cloud-based database 30. A protocol is based on general and patient specific clinical guidelines and adapted to a given patient, as described further hereinbelow. Audio content may include sounds, words and sentences presented. Visual content may include objects or text that the patient is expected to visually or auditory recognize and to express verbally. Activities are intended to integrate patient training into an attractive visual and audio experience, which is also typically custom designed according to a patient's specific characteristics, such as age and interests. In some embodiments, the patient application may be a web-based application or plug-in, and the protocol scripts may be code, such as HTML Java applet or Adobe™ Flash (SWF) code.

In embodiments, during a patient therapy session, the patient application 54 may send audio, visual, and intraoral signals from the patient to a progress module 60. The progress module may perform one or more assessment tests to determine the patient's progress in response to the protocol-based activities. As described in more detail below, assessments may include analyzing a patient's speech with respect to acoustic models.

Typically, protocol scripts 58 rely on assessments by the progress module 60 to determine how to proceed during a session and from session to session, for example, whether to continue with a given exercise or to continue with a new exercise.

The assessments by the progress module 60 also determine feedback to give a patient. Feedback may include accolades for being successful, or instructions regarding how to correct or improve the pronunciation of the sound or word. Feedback may be visual, audible, tactile or a combination of all three forms. When a patient masters pronunciation of a specific sound or word, the protocol may be configured to automatically progress to the next level of therapy.

Assessments are also communicated to the database system 30, which may be co-located with the patient platform but is more often remotely located and configured to support multiple remote patients and clinicians. The therapy data system may store the assessments with the patient records system 70

The patient application 54 may also pass speech, as well as other I/O device data, to a behavior module 62, which may identify whether a patient's behavior during a session indicates a need to modify the protocol of the session. The behavior module 62 may operate as a decision engine, with rules that may be generated by a machine learning process. The machine learning process is typically performed by the database system 30, and may include, for example, structured machine learning based on clustering algorithms or neural networks.

The behavior module 62 may be trained to identify, for example, a need to intervene in a custom protocol generated for a patient, according to parameters of behavior measured in various ways during a speech therapy session. Parameters may include, for example, a level of patient motion measured by the cameras 48, or a time delay between a prompt by an activity and a patient's response. Rules of the behavior module typically include types of behavior parameters and behavior thresholds, indicating when a given behavior requires a protocol intervention. Upon detecting that a behavior parameter exceeds a given threshold, the behavior module may indicate to the patient application 54 the type of intervention required, typically by modifying visual and/or audio aspects of the therapy session activity.

If, for example, a patient's ability to repeat words correctly drops from a rate of two trials per word to an average of five trials per word, the behavior module 62 may signal the patient application 54 to reduce a level of quality required for the patient's pronunciation to prevent patient frustration from increasing. If a patient requires too much time to identify objects on the display, that is, a patient's response delay surpasses a threshold set in the behavior module, the behavior module 62 may signal the patient application to increase the size of objects displayed. The behavior module 62 may also receive and analyze video from cameras 48, and may determine, for example, that a patient's movement indicates restlessness. The behavior module may be set to respond to a restlessness indication by changing an activity currently presented by the patient application.

The behavior module 62 may be based on an AI algorithm configured by generalizing patient traits, such as a patient's age, to determine how and when to change a protocol. For example, the behavior module 62 may determine after only one minute a need to modify visual or audio output for a child, based on behavior of the child, whereas the behavior module would delay such a modification (or, “intervention”) for a longer period for an adult patient. One purpose of the behavior module is to prevent a patient from being frustrated to an extent that the patient stops focusing or stops a session altogether. The behavior module 62 may determine that a child, for example, needs an entertaining break for a few minutes. Alternatively, the behavior module may determine a need to pop up a clinician video clip that explains and reminds the patient how to correctly pronounce a sound that is problematic.

The behavior module 62 may also determine from certain behavior patterns that a clinician needs to be contacted to intervene in a session. The patient platform 25 may be configured to send an alert to a clinician through several mechanisms, such as a video call or message. The alert mechanism enables a clinician to monitor multiple patients simultaneously, intervening in sessions as needed.

A clinician control module 64 of the patient platform 25 allows the clinician, working from the clinician interface 35 to communicate remotely with a patient, through the audio and/or video output of the patient platform. In addition, the clinician control module may allow a clinician to take over direct control of the patient application, either editing or circumventing the protocol script for a current session. The clinician interface 35 may be configured to operate from either a desktop computer or a mobile device. A clinician may decide to operate the remote communications either on a pre-scheduled basis, as part of a pre-planned protocol, or on an ad hoc basis. A clinician may also use the clinician interface 35 to observe a patient's session without interacting with a patient. When the patient is a minor, the parents interface 37 may be provided to allow the patient's parent or guardian to track the patient's progress through the treatment cycle. This interface also serves as a communication platform between the clinician and the parent for the purpose of occasional updates, billing issues, questions and answers, etc.

The clinician interface 35 also allows a clinician to interact with the database 30.

In embodiments, the database system 30 is configured to enable a clinician to register new patients, entering patient trait data into the patient records system. Patient trait data may include traits such as age and interests, as well as aspects of the patient's speech impediments. The clinician may also enter initial protocol scripts for a patient, a process that may be facilitated by a multimedia editing tool. Alternatively, based on the patient data, an analytics engine 72 of the therapy data system may determine a suitable set of protocols scripts 58 for a patient, as well as suitable behavior rules for the behavior module 62.

Processing by the analytics engine 72 may rely on a rules system, whose rules may be generated by a machine learning process, based on data in the therapy repository 74. Data in the therapy repository 74 typically includes data from previous and/or external (possibly global) patient cases, including patient records, protocols applied to those patients, and assessment results. The machine learning process determines protocols that are more successful for certain classifications of patients and speech impediments, and creates appropriate rules for the analytics engine.

Initial protocols and behavior rules are stored with the patient's records in the patient records system 70, and may be edited by the clinician. During a subsequent therapy session, the database system 30 also tracks a patient's progress, as described above, adding assessment data, as well as other tracking information, such as clinician-patient interaction, in the records system 70. In some embodiments, audio and/or video recordings of patient sessions may also be stored in the records system 70. As a patient progresses from session to session, the clinician can review the patient's progress from the patient's records, and may continue to make changes to the protocols and/or generate protocols or protocol recommendations from the analytics engine 72.

As described above, the database system 30 may be configured to support multiple remote patients and clinicians. Access to multiple clinicians and/or patients may be controlled by an operator through an operator interface 40. Security measures may be implemented to provide an operator with access to records without patient identifying information to maintain patient confidentiality. An operator may manage the therapy repository, transferring patient records (typically without identifying information) to the repository to improve the quality of data for the machine learning process and thereby improve the analytics engine 72.

The process of improving the analytics engine 72 can thus be seen as being a cyclical process. The analytics engine is first generated, typically by a machine learning process that extracts from patient records correlations of patient traits (age, language, etc.), speech disorders, and applied protocols, with progress metrics. This process generates rules that correlate speech impairments and patient traits to recommended protocols for automated speech therapy. The recommend rules are applied to a new patient case to create a custom protocol for the new patient, given the new patient's specific traits and speech impairments. The patient then participates in sessions based on the custom protocol, and the patient's progress is monitored. A metric of the patient's progress is determined, and depending on the level of the progress, the rules of the analytics engine may be improved according to the level of success obtained by the custom protocol.

In some embodiments, the therapy data system is a cloud-based computing system. Elements described above as associated with the patient platform, such as the progress and behavior modules, may be operated remotely, for example at the therapy data system itself.

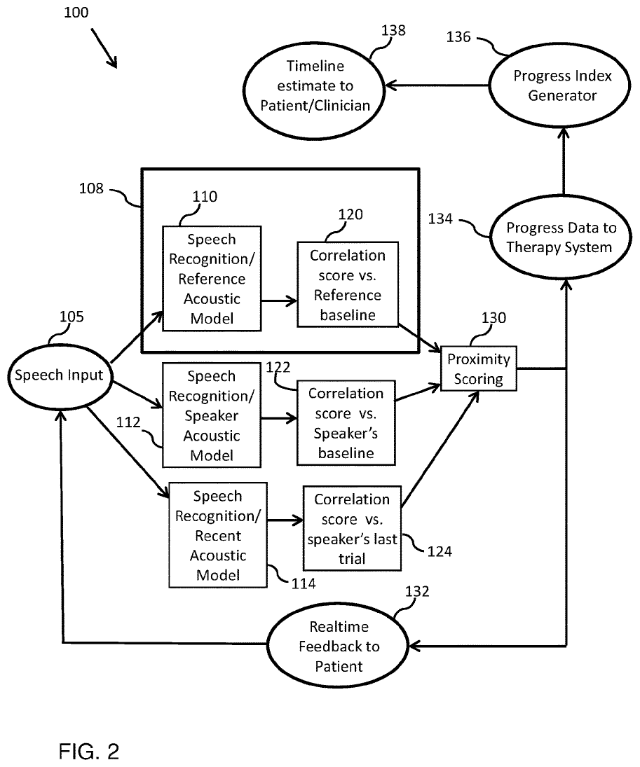

FIG. 2 is a schematic, flow diagram of a process 100 for progress assessment, implemented by the progress module 60, according to an embodiment. At steps 110 and 112, speech input 105 from a patient is processed by parallel, typically simultaneous, respective speech recognition processes. The process of step 110 compares the speech input with an acoustic model based on the patient's own patterns of speech (measured at the start of therapy, before any therapy sessions). The process of step 112 similar compares the speech input with an acoustic model based on a reference speaker (or an “ideal” speaker), that is, a speaker with speech patterns that represent a target for the patient's therapy. In some embodiments, speech of the reference speaker may also be the basis for audio output provided to the patient during therapy session. The acoustic models for any given assessment may be general models, typically based on phonemes, and/or may be models of a specific target sound, word or phrase that a patient is expected to utter for the given assessment. By definition, an acoustic model is used in automatic speech recognition to represent the relationship between an audio signal and the phonemes or other linguistic units that make up speech. In embodiments, progress of a patient is indicated by the respective correlations of a patient's utterances and the two acoustic models described above.

The output data of steps 110 and 112 are processed, at respective steps 120 and 122, to provide correlations scores. Step 120 provides a score correlating the patient's utterances throughout a therapy session with the patient's acoustic models. Step 112 correlates the patient's utterances with the reference speaker acoustic model at the step. The two scores are compared at a step 130, to provide a “proximity score”, which may be a score of one or more dimensions. In some embodiments, the proximity score may be generated by a machine learning algorithm, based on human expert evaluation of phoneme similarity, i.e., correlation. A perfect correlation with the patient's acoustic model may be normalized as 0, on a scale of 0 to 100, while a perfect correlation with the reference speaker acoustic model may be set to 100. Partial correlations with both models may be translated to a score on the scale of 0 to 100.

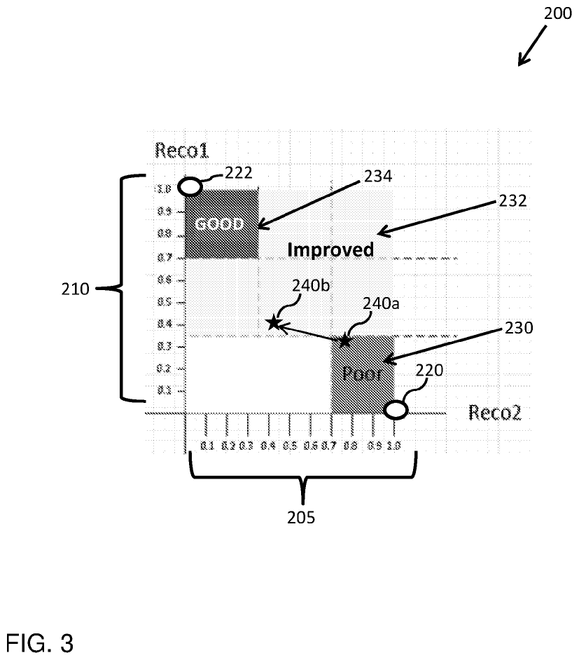

Alternatively, or additionally, the proximity score may be represented on a multi-dimensional (two or more dimension) graph, with axes represented by the correlation scores, as described further below with respect to FIG. 3. Regions of the graph may be divided into sections denoting “poor”, “improved”, and “good” progress.

The proximity score may also have a third component, based on a correlation of a patient's speech, at steps 114 and 124, with an acoustic model based on the patient's speech during a recent (typically the most recent) session. This aspect of the testing can show whether there are immediate issues of changes in the patient's speech that need to be addressed. In addition, the correlation determined at step 124 may indicate whether the equipment may be operating incorrectly. The correlation may be compared with a preset threshold value to indicate an equipment problem.

Based on the proximity score, which represents a metric of the patient progress, the patient application provides a visual and/or audio indication to the patient, at a step 132, representing feedback related to the patient's progress in improving his or her pronunciation. The feedback may be, for example, display of the proximity score, or sounding of a harmonious sound for good progress (e.g., bells), or a non-harmonious sound for poor progress (e.g., honking). Typically the feedback is provided in real time, immediately after the patient has verbally expressed the syllable, word, or phrase expected by the interactive activity.

In some embodiments, the level of progress, as well as encouragement and instructions, may be conveyed to the patient through an “avatar”, that is, an animated character appearing on the display 40 of the patient application 54. The avatar may represent an instructor or the clinician. In further embodiments, the motions and communications of the avatar may be controlled by a live clinician in real time, thereby providing a virtual reality avatar interaction.

In further embodiments, the proximity score (that is, an indicator or the patient's progress) may be also transmitted, at a step 134, to the cloud database system 30. Over the course of one or more therapy sessions, a patient's speech is expected to gradually have less correlation with the patient's original speech recognition acoustic model and more correlation with the reference speaker's speech recognition acoustic model. The patient's progress, maintained at the cloud database system, is available to the patient's clinician.

In addition, the analytics engine 72 of the database system 30 may be configured to apply machine learning processing methods, such as neural networks, to determine patterns of progress appearing across multiple patient progress records, thereby determining a progress timeline estimation model. As a given patient proceeds with therapy sessions, his or her progress may be compared, or correlated with, an index provided by the timeline estimation model, accounting for particular features of the given patient's problems and prior stages of progress. The comparison will provide an estimation of a timeframe for future progress or speed of pronunciation acquisition. In some embodiments, the analytics engine may be supplied with a sufficient number of patient records, to provide a global index. The model may also account for patient characteristics such as language basis, country, age, gender, etc. In further embodiments, the expected timeframe can also be associated with appropriate lessons for achieving target goals within the timeframe provided by the estimate. Consequently, the system will provide an economical and efficient means of designing an individualized course of therapy.

FIG. 3 is a schematic graph 200 of therapy assessment output, according to an embodiment. As described above, speech of a patient may be assessed by a dual correlation metric, including a first correlation between an utterance of the patient and an acoustic model of the patient's speech, at the start of therapy (before therapy sessions), and a second correlation between an utterance of the patient and an acoustic model of a reference speaker. Such a metric may be graphed in two dimensions, a first dimension 205 representing the correlation to the patient's acoustic model, and the second dimension 210 representing the correlation to the reference speaker's acoustic model. A point 220, at one extreme point of the graph, represents correlation of the patient's speech to his own acoustic model, at the beginning of therapy. A point 222, at an opposite extreme point of the graph, represents a perfect correlation to the reference speaker acoustic model.

Assuming that the patient progresses, the patient's speech gradually shows less correlation to the patient's original acoustic model and more correlation with the reference speaker acoustic model, that is, a metric of the patient's speech should show an improvement that might be indicated, for example, by point 240 a on the graph, and subsequently by a point 240 b. Lack of improvement from a given point over time is an indication that a given protocol no longer is effective and that the protocol must be modified. Graph 200 may be divided into three or more sections, to indicate various ranges of improvement. For example, a correlation of 0.7 or more with the patient acoustic model and of 0.35 or less with the reference speaker acoustic model is indicated as a “poor” improvement region 230. A correlation of 0.35 or better with the reference speaker acoustic model is indicated as an “improved” region 232. A correlation of 0.7 or more with the reference acoustic model, and of 0.35 or less with the patient speaker acoustic model, is indicated as a “good” improvement region 234.

It is to be understood that the embodiments described hereinabove are cited by way of example, and that the present invention is not limited to what has been particularly shown and described hereinabove. The rules engines described above may be developed by methods of machine learning such as decision trees or neural networks. The classification of speech scores may include further sub-classifications to distinguish types of difficulties. Additional changes and modifications, which do not depart from the teachings, will be evident to those skilled in the art. Computer processing elements described may be distributed processing elements, implemented over wired and/or wireless networks. Such computing systems may furthermore be implemented by multiple alternative and/or cooperative configurations, such as a data center server or a cloud configuration of processers and data repositories. Processing elements of the system may be implemented in digital electronic circuitry, or in computer hardware, firmware, software, or in combinations thereof. Such elements can be implemented as a computer program product, tangibly embodied in an information carrier, such as a non-transient, machine-readable storage device, for execution by, or to control the operation of, data processing apparatus, such as a programmable processor, computer, or deployed to be executed on multiple computers at one site or distributed across multiple sites. Memory storage may also include multiple distributed memory units, including one or more types of storage media.

Communications between systems and devices described above are assumed to be performed by software modules and hardware devices known in the art. Processing elements and memory storage, such as databases, may be implemented so as to include security features, such as authentication processes known in the art.

Method steps associated with the system and process can be rearranged and/or one or more such steps can be omitted to achieve the same, or similar, results to those described herein. The scope includes variations and modifications thereof which would occur to persons skilled in the art upon reading the foregoing description and which are not disclosed in the prior art.

### Other Examples of Solutions Related to Speech Therapy

### AI Based Solution

There are provided a solution (any one of a systems and/or a method and/or a non-transitory computer readable medium that stores instructions for—or a combination thereof) using physiological, visual and vocal parameters to analyze and detect tiredness or loose of interest in the treatment and practice.

These parameters will allow the solution to determine what is the current mental and physical condition of the user (high/low interest in the practice, attention, focusing, awareness, etc.) to fit the treatment (for example in an optimal way), when to stop, when to practice, increasing/decreasing of hardness level, etc.

The solution may use a AI based process that is configured to analyze and evaluate in real time these parameters and change the course of treatment adopting to the patient's mental and physiological state.

### G.O.M—Goodness of Movement.

A part of the pronunciation is the mechanical movement of the mouth, jaw, teeth, tongue, throat. There may be provided a process for listening to the way the person pronounces the words or sentences. Additionally or alternatively, there may be provided another process that analyzes the mechanical movement of the mentioned above and is configured to provide a better understanding whether the pronunciation is correct, and if not, why not.

The measurement of the speech production is done not only by the voice produced by the patient, but also by the correctness of the motoric movement of the mouth.

This will be achieved by tracking the mouth movement by the mobile device's camera in real time.

### Visual Feedback.

The visual feedback may be provide in addition to the G.O.P solution—for example in case the person pronounces the word/sentence wrongly, after several repeats in a wrong way (number of repeats can be configurable). In such a case the process will raise a flag with inputs and the system will initiate a short animation or a real short movie of how to perform the mechanical movement to get the correct pronunciation.

### Privacy Safeguard.

There may be a need to see relevant parts of the person (for example jaw) while masking other parts of the person—to maintain confidentiality.

This may be obtained by face masking to keep the user privacy—as part of the privacy protection of the user, when the user uses the solution, the solution records a video of the practice (voice and visual) so the clinician can review it later, but may mask some parts of the face of the user, leaving only the nose, mouth, neck and shoulders visible.

The mask dynamically is adjusted to movements of the user in order not to expose the entire face of the user.

### Enhancing Online Synchronic Treatment.

Some treatments may be (a) Face to Face personal treatment in the clinic—the clinician treats the patient in the clinic, (b) Face to Face online personal treatment—the clinician treats the patient using an online SW tool (like zoom), (c) Face to Face group treatment in clinic—the clinician sit with more than one patient in the clinic and treat them together (time splits between the patients evenly), or (d) Face to Face online group treatment—patients can sit, either together (in class, for example) or separately (at home, for example) and the clinician sit separately from the patients (in her/his clinic, for example) and treat the group together. Each patient will get even time like the others, they all see and hear the other patients.

Options (c) and (d) are inefficient due to the short time the patient has to work and practice with the clinician. That leads to a long and frustrating process of treatment which, sometime ends without results due to the long and inefficiency of the process.

There may be provided a solution that displays a practitioner to conduct multi session simultaneously in a way each patient, regardless of his location, is an independent window that can see and hear only the clinician.

The solution calculates, for one, more or all patients, a score or any other indication regarding the quality of pronunciation or of the practice of—and may generate alerts (audio and/or visual) indication which patients need assistance/which patients need more assistance than other, where the quality of practice is below a threshold, and the like.

The score may be generated by a correctional feedback process (a process that listen to the patient talks and gives him or her a score that can be either a number (for example 1-100) or a color (for example, green, orange, red) that reflects the goodness of the pronunciation, and/or quality of practice—and the score is reflected to the clinician on his screen (clinician sees multi screens and the results on each screen) allows the clinician to work simultaneously with more than one patient and focus on the patients who really need assistant. For example, if the clinician works with 4 patients simultaneously and according to the scores reflected on his screens, patients 1 and 4 getting high scores whereas patients 2 and 3 get low scores, he can focus his time on patients 2 and 3 and leave patients 1 and 4 to continue practice without interferences or change their protocol to practice new material, new sounds or word/sentences, increase the hardness level of practice or in the best case decide that the treatment is no longer required.

### Remote Diagnostic of Speech and Language Pathologies.

Various diagnostics are being performed in a Face-to-Face model. That consume time of the patient and parents, minimize the availability of the clinician, delay the diagnostic process due to lack of clinicians and long waiting lists.

The usage of video camera with a microphone and devices that can either be placed near the patient or on the patient (neck, for example) that generate electro-magnetic resonance (like in MRI), or an accelerometer that feels the movements and directions can be used to determine the goodness of pronunciation as well as the mechanical movements associated with the performed sound and diagnose the problem and come up with the proper treatment protocol.

The remote diagnostic may also be based on standard diagnostic protocols.

The ability of the solution to evaluate the correctness of the pronunciation of each phoneme in a word or a sentence will allow for the remote assessment session which will produce recommendations for the needed and personalized treatment plan.

This application provides a significant technical improvement over the prior art—especially an improvement in computer science.

Any reference to the term “comprising” or “having” should be applied mutatis mutandis to “consisting” and/or should be applied mutatis mutandis to “essentially consisting of”. For example—a method that comprises certain steps can include additional steps. A method that consists certain steps can be limited to the certain steps. A method that essentially consists of certain steps may include the certain steps as well as additional steps that do not materially affect the basic and novel characteristics of the method.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a computer program product such as non-transitory computer readable medium. All or some of the computer program may be provided on computer readable media permanently, removably or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as FLASH memory, EEPROM, EPROM, ROM; ferromagnetic digital memories; MRAM; volatile storage media including registers, buffers or caches, main memory, RAM, etc. A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system. The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device. Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A non-transitory computer readable medium for real time evaluating a mechanical movement associated with a pronunciation of a phenome by a patient, the non-transitory computer readable medium stores instructions that once executed by a processing circuit cause the processing circuit to:
receive visual information regarding a patient mechanical movement that is associated with the pronunciation of the phenome by the patient;
apply a machine learning based extraction process for extracting features of the patient mechanical movement;
determine, by a classifier and based on the features, a quality of the patient mechanical movement; wherein the classifier was trained by a training process that comprises feeding to the classifier with features of examples of visual mechanical movement information, wherein the examples are associated with a quality score that is determined based on speech therapy experts feedbacks; and
respond to the quality of the patient mechanical movement.

2. The non-transitory computer readable medium according to claim 1 wherein the responding comprising participating in a transmission of mechanical movement feedback to the patient.

3. The non-transitory computer readable medium according to claim 2 wherein the mechanical movement feedback comprises a patient provided quality score.

4. The non-transitory computer readable medium according to claim 3, wherein the mechanical movement feedback also comprises information about how to correct the patient mechanical movement, when the patient provided quality score is indicative of a faulty.

5. The non-transitory computer readable medium according to claim 1 wherein the visual information comprises a clear segment of a face of the person, the clear segment covers at least a part of a mouth of the patient and at least a part of a vicinity of the mouth of the patient, the vicinity does not include eyes of the patient.

6. The non-transitory computer readable medium according to claim 5 wherein the visual information also comprises an unclear representation of eyes and nose of the patient.

7. The non-transitory computer readable medium according to claim 1 wherein the classifier is a machine learning classifier.

8. The non-transitory computer readable medium according to claim 1 wherein the classifier differs from a machine learning classifier.

9. The non-transitory computer readable medium according to claim 1 wherein the examples received a same speech therapy experts feedback from all speech therapy experts.

10. The non-transitory computer readable medium according to claim 1 wherein the examples received a same speech therapy experts feedback from a majority of speech therapy experts.

11. The non-transitory computer readable medium according to claim 1 wherein the evaluating of the mechanical movement is responsive to a location of the phenome within a word.

12. The non-transitory computer readable medium according to claim 11 that stores instructions that once executed by a processing circuit cause the processing circuit to select the machine learning based extraction process, out of a plurality of machine learning based extraction process associated with different locations of the phenome, based on the location of the phenome within the word.

13. The non-transitory computer readable medium according to claim 12 that stores instructions that once executed by a processing circuit cause the processing circuit to select the classifier, out of a plurality of classifiers associated with different locations of the phenome, based on the location of the phenome within the word.

14. The non-transitory computer readable medium according to claim 1 that stores instructions that once executed by a processing circuit cause the processing circuit to obtain an indication of an audio quality of the pronunciation of the phenome by the patient.

15. The non-transitory computer readable medium according to claim 14 that stores instructions that once executed by a processing circuit cause the processing circuit to respond to the quality of the patient mechanical movement and to the indication of the audio quality.

16. The non-transitory computer readable medium according to claim 14 that stores instructions that once executed by a processing circuit cause the processing circuit to calculate the patient provided quality score based on the quality of the patient mechanical movement and the indication of audio quality.

17. The non-transitory computer readable medium according to claim 1 wherein the features of the patient mechanical movement are selected out of a group of candidate features of the patient mechanical movement.

18. The non-transitory computer readable medium according to claim 1 that stores instructions that once executed by a processing circuit cause the processing circuit to evaluate a mechanical movement associated with a pronunciation of another phenome by the patient.

19. A computer implemented method for real time evaluating a mechanical movement associated with a pronunciation of a phenome by a patient, the computer implemented method comprises:
receiving visual information regarding a patient mechanical movement that is associated with the pronunciation of the phenome by the patient;
applying a machine learning based extraction process for extracting features of the patient mechanical movement;
determining, by a classifier and based on the features, a quality of the patient mechanical movement; wherein the classifier was trained by a training process that comprises feeding to the classifier with features of examples of visual mechanical movement information, wherein the examples are associated with a quality score that is determined based on speech therapy experts feedbacks; and
responding to the quality of the patient mechanical movement.

20. The computer implemented method according to claim 19, wherein the responding comprising participating in a transmission of mechanical movement feedback to the patient.

21. The computer implemented method according to claim 20, wherein the mechanical movement feedback comprises a patient provided quality score.

22. The computer implemented method according to claim 21, wherein the mechanical movement feedback also comprises information about how to correct the patient mechanical movement, when the patient provided quality score is indicative of a faulty.

23. The computer implemented method according to claim 19, wherein the visual information comprises a clear segment of a face of the person, the clear segment covers at least a part of a mouth of the patient and at least a part of a vicinity of the mouth of the patient, the vicinity does not include eyes of the patient.

24. The computer implemented method according to claim 23, wherein the visual information also comprises an unclear representation of eyes and nose of the patient.

25. The computer implemented method according to claim 19, wherein the classifier is a machine learning classifier.

26. The computer implemented method according to claim 19, wherein the classifier differs from a machine learning classifier.

27. The computer implemented method according to claim 19, wherein the examples received a same speech therapy experts feedback from all speech therapy experts.

28. The computer implemented method according to claim 19, wherein the examples received a same speech therapy experts feedback from a majority of speech therapy experts.

29. The computer implemented method according to claim 19, wherein the evaluating of the mechanical movement is responsive to a location of the phenome within a word.

30. The computer implemented method according to claim 29, comprising selecting the machine learning based extraction process, out of a plurality of machine learning based extraction process associated with different locations of the phenome, based on the location of the phenome within the word.

31. The computer implemented method according to claim 30, comprising selecting the classifier, out of a plurality of classifiers associated with different locations of the phenome, based on the location of the phenome within the word.

32. The computer implemented method according to claim 19, comprising obtaining an indication of an audio quality of the pronunciation of the phenome by the patient.

33. The computer implemented method according to claim 32, comprising responding to the quality of the patient mechanical movement and to the indication of the audio quality.

34. The computer implemented method according to claim 33, comprising calculating the patient provided quality score based on the quality of the patient mechanical movement and the indication of audio quality.

35. The computer implemented method according to claim 19, wherein the features of the patient mechanical movement are selected out of a group of candidate features of the patient mechanical movement, based on statistical significance.

36. The computer implemented method according to claim 19 comprising evaluating a mechanical movement associated with a pronunciation of another phenome by the patient.

37. A computerized system for real time evaluating a mechanical movement associated with a pronunciation of a phenome by a patient, the computerized system comprises one or more processing circuits that are configured to:
receive visual information regarding a patient mechanical movement that is associated with the pronunciation of the phenome by the patient;
apply a machine learning based extraction process for extracting features of the patient mechanical movement;
determine, by a classifier and based on the features, a quality of the patient mechanical movement; wherein the classifier was trained by a training process that comprises feeding to the classifier with features of examples of visual mechanical movement information, wherein the examples are associated with a quality score that is determined based on speech therapy experts feedbacks; and
respond to the quality of the patient mechanical movement.
